(19) United States
(12) Patent Application Publication
(10) Pub. No.: US 2026/0033824 A1
(43) Pub. Date: Feb. 5, 2026

(54) KNOTLESS ANCHOR SYSTEM AND METHOD

(71) Applicant: AEVUMED, INC., Malvern, PA (US)

(72) Inventors: Miles O. Curtis, Philadelphia, PA (US); Saif Khalil, Malvern, PA (US)

(21) Appl. No.: 19/286,727

(22) Filed: Jul. 31, 2025

Related U.S. Application Data
(60) Provisional application No. 63/678,443, filed on Aug. 1, 2024.

(51) Int. Cl. A61B 17/04; A61B 17/16

(52) U.S. Cl. CPC A61B 17/0401; A61B 2017/0409; A61B 2017/0414; A61B 17/1622

(57) ABSTRACT

A suture anchor system is disclosed. The system includes at least one suture anchor having an elongate anchor body having at least one anchoring element, and a channel having a distal channel loop and a proximal opening in a proximal face of the elongate anchor body. The system also includes a tapered repair suture. A method for treating soft tissue is also disclosed.

### CROSS-REFERENCE TO RELATED APPLICATIONS

This application claims priority to U.S. Provisional Application No. 63/678,443 filed on Aug. 1, 2024, incorporated herein by reference in its entirety.

### BACKGROUND OF THE INVENTION

Knotless suture anchors are used in various surgical procedures including orthopedic and soft tissue surgeries to anchor and secure soft tissue (such as tendons or ligaments) to bone. These anchors provide a secure fixation point for sutures, which are used to reattach or repair damaged tissue. Knotless suture anchors are commonly used in procedures like rotator cuff repairs, labral repairs in the shoulder, Achilles tendon repairs, and ligament reconstructions.

Existing knotless suture anchors have inconsistent suture tensioning and fixation strength, and under cyclic loading, suture can slip or pull out of the anchor. Further, existing anchors lack feedback to the surgeon during tensioning, and confirmation that an optimal tension has been reached.

Thus, there is in the need in the art for improved knotless suture anchor systems and methods that provide consistent tensioning and fixation strength, as well as provide feedback to the surgeon during reparative procedures. The present invention meets this need.

### SUMMARY OF THE INVENTION

In one embodiment, a suture anchor system includes at least one suture anchor having an elongate anchor body having at least one anchoring element, and a channel having a distal channel loop and a proximal opening in a proximal face of the elongate anchor body, and a tapered repair suture. In one embodiment, the tapered repair suture is configured to arrest advancement of the repair suture via an interface between a tapered portion of the tapered repair suture and the channel. In one embodiment, the at least one suture anchor comprises an eyelet extending through a portion of the anchor body. In one embodiment, the tapered repair suture is preloaded through the eyelet. In one embodiment, the tapered repair suture is affixed to the eyelet. In one embodiment, the suture anchor system includes at least one shuttle suture. In one embodiment, the at least one shuttle suture is preloaded through the distal channel loop of the suture anchor. In one embodiment, the at least one shuttle suture comprises a looped end and a free end, each extending out from the proximal opening of the anchor body. In one embodiment, the tapered repair suture releasably engages the looped end of the shuttle suture. In one embodiment, the at least one suture anchor includes first and second suture anchors. In one embodiment, the tapered suture tapers in width along its length from about 0.3 mm to about 0.65 mm. In one embodiment, the tapered suture has a length ranging between 80 mm and 200 mm. In one embodiment, the suture anchor system incudes a guide tool; and an inserter tool, wherein the suture anchor engages the inserter tool, and the inserter tool extends through a portion of the guide tool to secure the suture anchor in a bone site. In one embodiment, the suture anchor system includes a drill tool, wherein the drill tool extends through a portion of the guide tool to drill a drill hole at the bone site.

In one embodiment, a method for treating soft tissue includes the steps of securing the suture anchor into a hole in bone; passing the tapered repair suture at least partially around the soft tissue; shuttling the tapered repair suture through the distal channel loop; and cinching the tapered repair suture in the channel of the anchor, thereby capturing the soft tissue with the tapered repair suture. In one embodiment, the tapered repair suture is affixed to the suture anchor prior to the securing step. In one embodiment, the method includes providing a second suture anchor; securing the second anchor into a second hole in bone; passing the tapered repair suture at least partially around the soft tissue; shuttling the tapered repair suture through the distal channel loop of the second suture anchor; and cinching the tapered repair suture in the channel of the second suture anchor, thereby capturing the soft tissue with the tapered repair suture. In one embodiment, the method includes securing the suture anchor into a hole in bone; passing the tapered repair suture at least partially around the soft tissue; shuttling the tapered repair suture through the distal channel loop of the anchor with the shuttle suture; and cinching the tapered repair suture in the channel of the suture anchor, thereby capturing the soft tissue with the tapered repair suture. In one embodiment, the method includes releasing the shuttle suture from the tapered repair suture. In one embodiment, the method includes providing a second suture anchor; securing the second suture anchor into a second hole in bone; passing the tapered repair suture at least partially around the soft tissue; shuttling the tapered repair suture through the distal channel loop of the second suture anchor; and cinching the tapered repair suture in the channel of the second suture anchor, thereby capturing the soft tissue with the tapered repair suture.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIGS. 1A-1D depict views of an exemplary suture anchor (e.g., a knotless suture anchor) according to one embodiment. FIG. 1A depicts a side view of an exemplary suture anchor. FIG. 1B depicts a tapered repair suture according to one embodiment. FIG. 1C depicts side views of an exemplary suture anchor loaded with a tapered repair suture (left), that expands in width as it is pulled through the anchor (right). FIG. 1D depicts an exemplary suture anchor being used with a shuttle suture and a tapered repair suture for tissue repair.

FIGS. 2A-2D depict various views of an exemplary system (e.g., a knotless suture anchor system). FIG. 2A depicts an exemplary system comprising components such as a suture anchor, one or more sutures (e.g., a shuttle suture and a tapered repair suture), a guide tool, and an inserter tool. FIG. 2B depicts components of an exemplary system further comprising a drill that may extend through the guide. FIG. 2C depicts an enlarged view of the distal ends of exemplary guide tool and inserter tool, with a suture anchor engaged with the inserter tool. FIG. 2D depicts an enlarged view of the distal ends of an exemplary guide tool and inserter tool, with the inserter tool extending through the guide tool.

FIGS. 3A-3B depict exemplary steps for a method of using a knotless suture anchor system. A guide tool with trocar is placed in bone (FIG. 3A) and a drill tool is inserted into the guide tool (FIG. 3B) to drill an anchor hole in the bone.

FIGS. 4A-4C depict exemplary steps for a method of using a knotless suture anchor system. The inserter tool is inserted into the guide tool (FIGS. 4A & 4B), and the head or handle of the inserter tool is impacted until the anchor stops and is fully advanced into the bone hole (FIG. 4C).

FIGS. 5A-5C depict exemplary steps for a method of using a knotless suture anchor system. Both the inserter tool and guide tool are withdrawn, leaving the anchor in the hole and tapered repair suture and shuttle suture exposed (FIG. 5A). The free end of the tapered repair suture is passed below the labrum (FIG. 5B). The free end of the tapered repair suture is then fed into the loop of the shuttle suture (FIG. 5C)

FIGS. 6A-6B depict exemplary steps for a method of using a knotless suture anchor system. The free end of the tapered repair suture is passed through the loop of the shuttle suture (FIG. 6A), and pulling the free end of the shuttle suture shuttles the tapered repair suture through the anchor (FIG. 6B).

FIGS. 7A-7B depict exemplary steps for a method of using a knotless suture anchor system. The tapered repair suture is looped around the labrum and through the anchor (FIG. 7A), and pulling the tapered repair suture cinches it into place. The taper in the tapered repair suture is designed to arrest movement of the suture in the anchor. A tight loop is formed (FIG. 7B) around the labrum, holding it securely against the anchor and bone.

FIG. 8 depicts an exemplary method of using a knotless suture anchor system.

### DETAILED DESCRIPTION

Embodiments of the knotless anchor system described herein utilize a suture anchor preloaded with a shuttling suture and a tapered repair suture for reattaching or repairing damaged tissue.

It is to be understood that the figures and descriptions of the present invention have been simplified to illustrate elements that are relevant for a more clear comprehension of the present invention, while eliminating, for the purpose of clarity, many other elements found in knotless anchor systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a knotless anchor system with suture anchor and method utilizing a tapered suture.

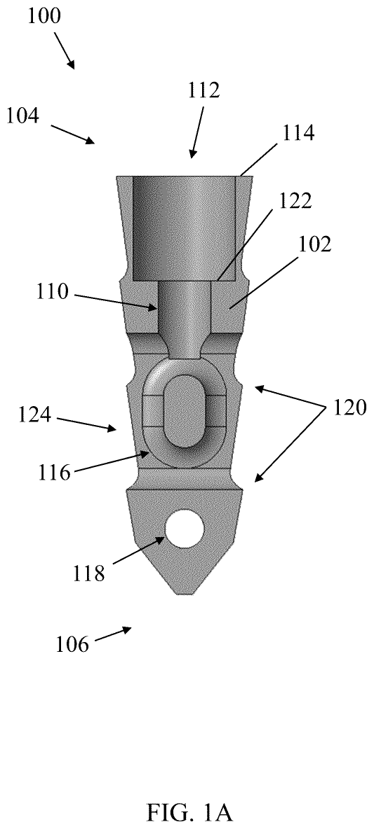

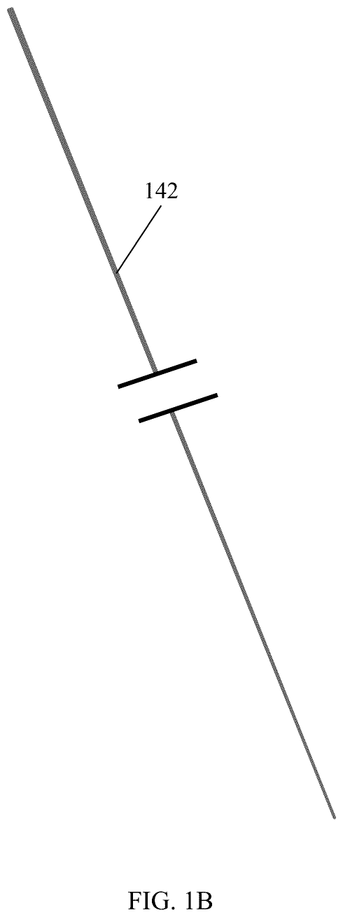

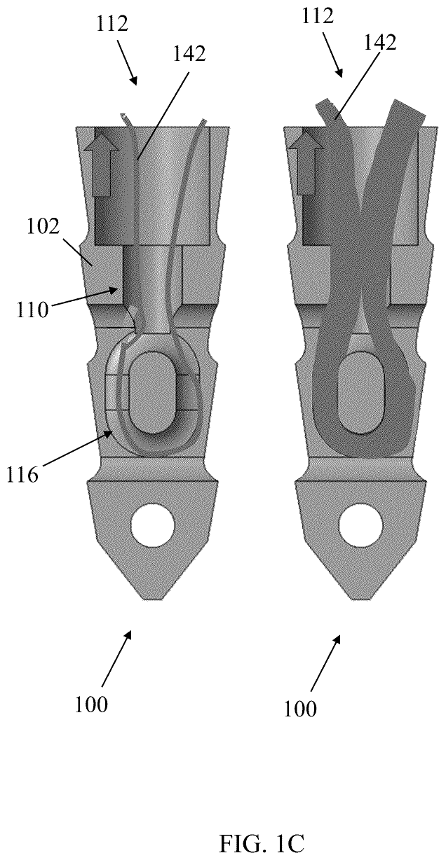

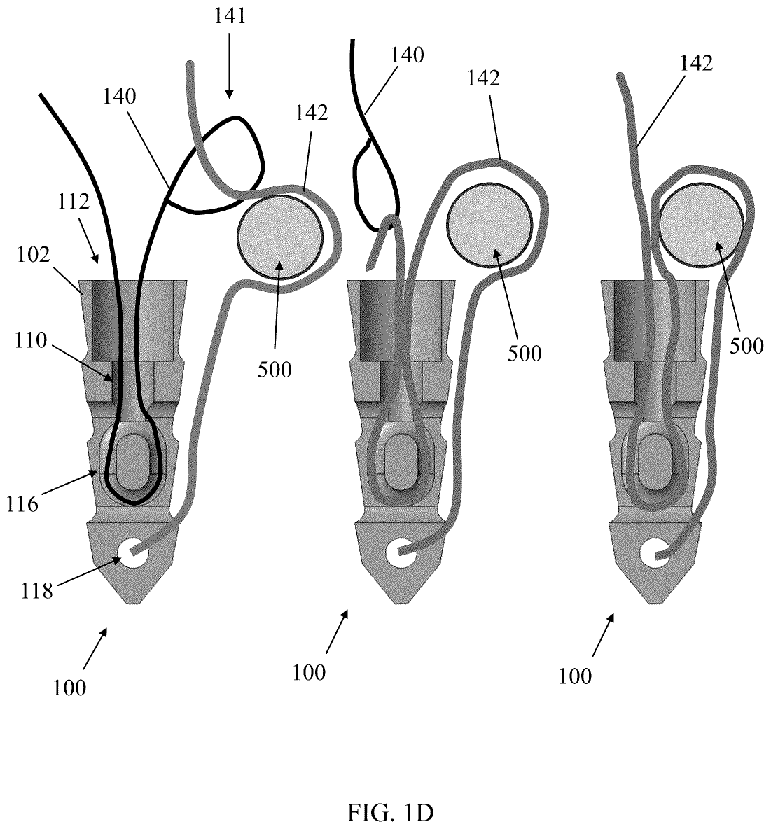

Referring now to FIG. 1A, shown is an exemplary suture anchor 100 comprising an elongate anchor body 102 having a proximal end 104 and a distal end 106. Suture anchor 100 comprises a channel 110 extending through at least a portion of body 102. In some embodiments, the channel 110 has an opening 112 in a proximal face 114 of body 102, and further has a channel loop 116 positioned in a portion of body 102 and distal portion of the channel 110. In some embodiments, suture anchor 100 comprises an eyelet 118 passing through at least a portion of body 102, for example laterally through a distal portion of body 102. As described herein, one or more lengths of suture may pass through portions of anchor 100, such as opening 112, channel 110, channel loop 116 and/or eyelet 118. In some embodiments, a shuttle suture pulls a tapered suture (e.g. FIG. 1D) through a portion of suture anchor 100, and cinches a wider portion of the tapered suture inside the anchor as the tapered suture becomes wider than the suture channel. The disclosed anchor may be used in conjunction with systems and methods for various types of reparative and soft tissue procedures as described further herein.

With additional reference now to FIG. 1C, shown are two views of an exemplary suture anchor 100 showing a tapered repair suture 142 being pulled through the anchor 100. Tapered suture 142 may have a tapered portion that expands moving longitudinally along the tapered suture's length, expanding in width (e.g. diameter or cross-sectional area) until the tapered suture can no longer advance through channels of the anchor. The expansion may for example be an expansion in diameter from 0.3 mm to 0.65 mm. The expansion may be for example over a particular tapered region of the tapered suture, where regions outside the tapered region are un-tapered or otherwise a constant width. For example, in one embodiment, the tapered region is only a proximal portion of the tapered suture. In one embodiment, only the last 100 mm of the suture expands in diameter, offering cinching between multiple anchors. The taper may for example be a constant taper or a variable taper, for example a taper that has a higher variable increase in diameter along different parts of the tapered portion. In one embodiment, a variable taper increases more rapidly moving towards the portion of the taper configured to cinch within the channel loop. In one embodiment a material change or coating is included on at least part of the tapered region of the suture to increase the lock and tight grip of the taper within the channel loop. The channel loop may also in certain embodiments feature a reverse-taper to counteract and interface with tapered region of the suture. Thus in certain embodiments, as the tapered suture is pulled through the channel loop, the tapered suture may increase in diameter as the channel loop decreases in diameter along that same direction, creating a wedged interface between the tapered suture and the channel loop. Other parts of the channel or other pathways of the anchor may alternately be utilized as the channel that interfaces with the tapered region.

As explained in further detail with reference to FIG. 1D, one or more of the shuttle sutures may be preloaded into suture anchor 100 for loading the tapered repair suture 142 through the channel loop 116. In some embodiments, one or more shuttle suture 140 and/or tapered suture 142 are preloaded through channel 110 and/or channel loop 116. In some embodiments, shuttle suture 140 is used to shuttle tapered suture 142 through suture anchor 100. In some embodiments, shuttle suture 140 comprises one or more ends with a loop or looped portion 141. In some embodiments, tapered suture 142 releasably engages the looped portion 141 of shuttle suture 140. It should be appreciated that shuttle suture 140 may be used to pull tapered suture 142 through channel 110 and channel loop 116 to cinch a tapered portion of tapered suture 142 within anchor 100, and then may be released from the system and/or discarded.

Further as shown for example in FIG. 1D, the diagram demonstrates the use of the anchor 100 for a labral repair procedure. The anchor 100 is configured to shuttle the tapered suture 142 (e.g., repair suture) though the anchor's channel loop 116 via the shuttle suture 140. As tapered suture 142 is pulled though channel 110 and channel loop 116, the suture initially moves freely. Upon increase of width and/or diameter of the suture, a portion of tapered suture 142 cinches within anchor 100. The tapered portion wedges in place by friction and other forces (e.g., radial, compressive) arresting further movement of tapered suture 142 within the channel 110. In some embodiments, the tapered suture 142 expands or tapers from 2-0 to #2 (0.3 mm to 0.65 mm) with a gradual transition. In some embodiments, tapered suture 142 has at least a first width or diameter ranging between 0.05 mm and 1 mm, and a second tapered width or diameter ranging between 0.1 and 2 mm. In some embodiments, the tapered suture material may have a level of compressibility, thus a portion of the channel 110 is smaller than the widest part of the tapered suture material to arrest movement of the tapered suture. A portion of the channel 110 (e.g. the channel loop 116) may be smaller than the widest part of the tapered suture material, such as about 90% smaller, 80% smaller, 70% smaller, 60% smaller, 50% smaller or more. A portion of the channel 110 can be configured sufficiently smaller than a tapered portion of the tapered suture based on the size and material used for the tapered suture. In some embodiments, tapered suture 142 comprises one or more tapered portions along its length in sections for affixing to more than one anchor. In some embodiments, the last 100 mm of the tapered suture 142 expands in diameter to enable remaining repair suture length for cinching between multiple anchors. The repair suture may also for example have multiple tapered regions, sized differently to correspond with channels of two or more anchors so that the same repair suture cinches at different portions along it's length within two or more different anchors.

In some embodiments, channel 110 and/or channel loop 116 are sized and shaped to allow shuttle suture 140 to freely pass through, however a wider or tapered portion of tapered suture 142 will eventually cinch within portions of either channel 110 and/or channel loop 116. In some embodiments, channel 110 comprises a circular or round cross-section, but channel 110 may be formed in any shape. In some embodiments, channel 110 tapers in width or diameter along its length. In some embodiments, channel 110 comprises friction enhancements such as ridges, high-friction coating, and the like. In some embodiments, portions of channel 110 and/or channel loop 116 may taper in height or width, or form one or more cinching regions. In some embodiments, channel loop 116 comprises anti-friction enhancements, such as anti-friction coating, fillets, and the like.

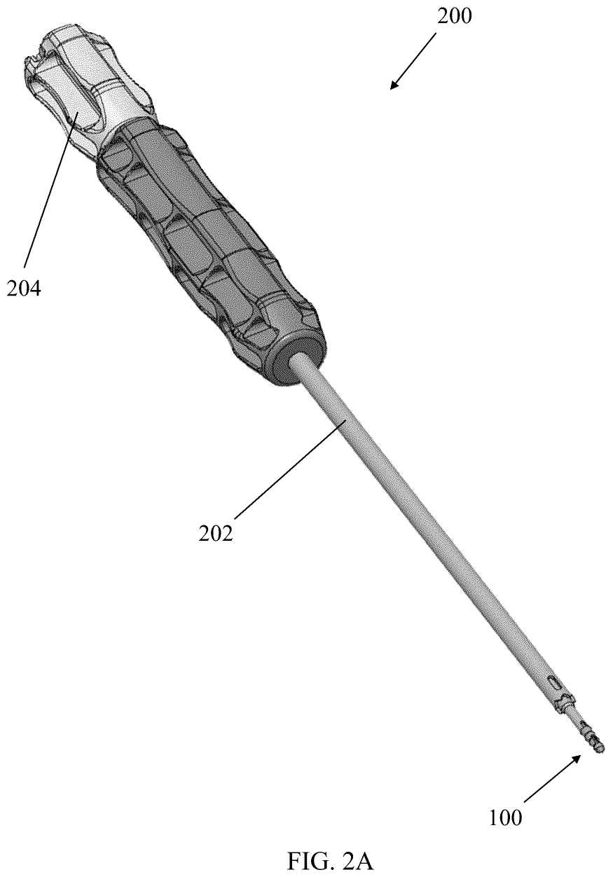

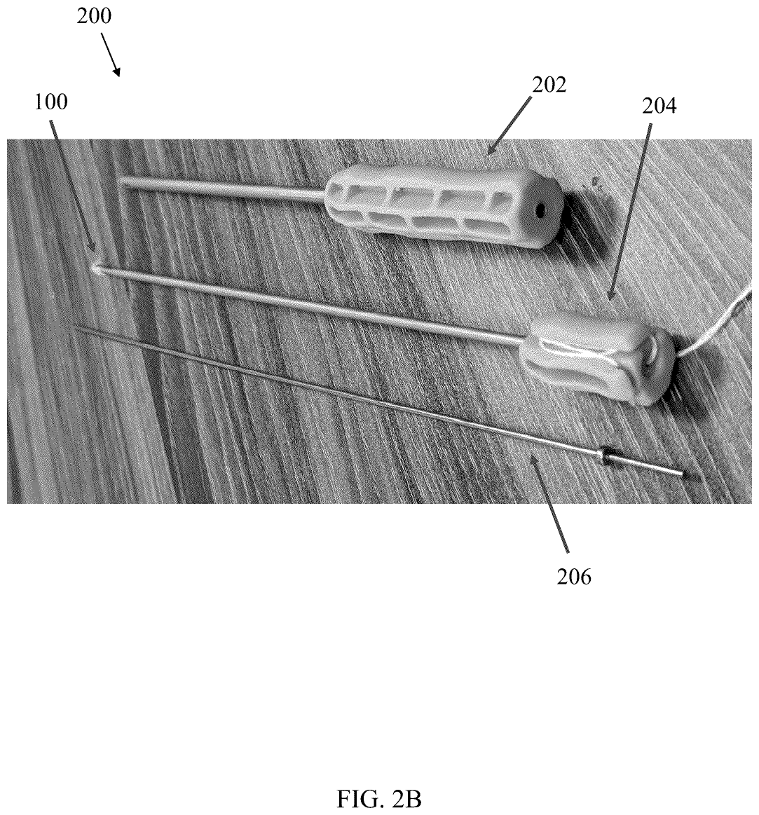

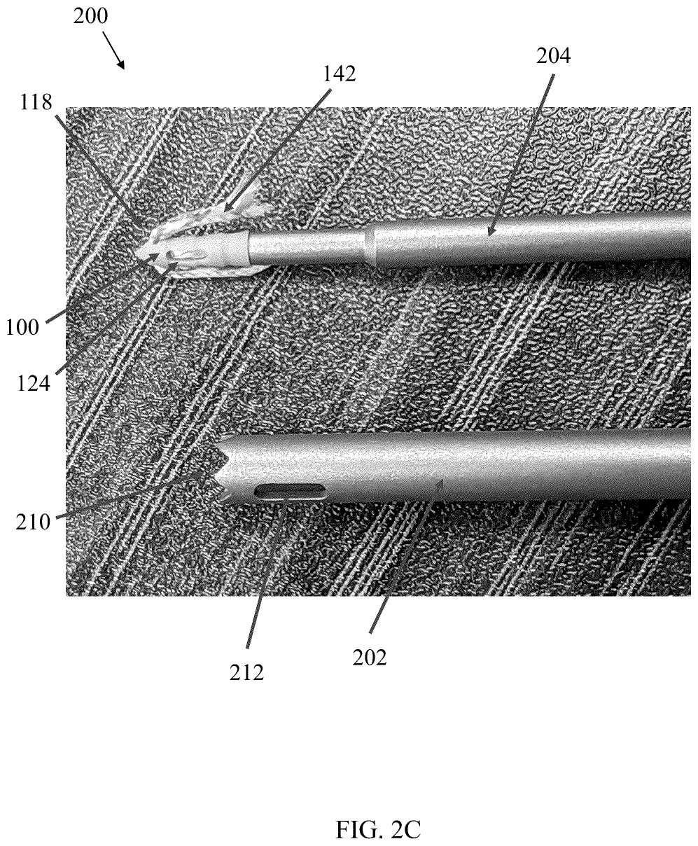

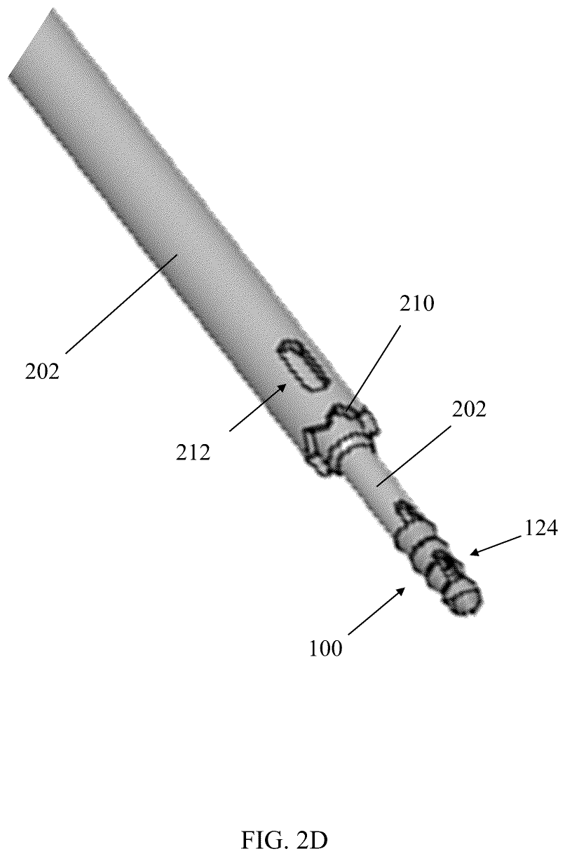

Suture anchor 100 may comprise any number of anchoring elements such as anchoring member 120 on body 102 to secure the anchor to a portion of a subject (e.g., into bone and/or a drill hole). Anchoring elements may comprise threads, barbs or ribs on body 102, with any combination of major and minor threads, or barbs, each having different or same diameters. The anchor material can be for example any material known in the art, including a medical grade polymer such as PEEK, or a medical grade metal such as titanium or stainless steel. Further, suture anchor 100 may further comprise any number of openings in body 102, such as one or more lateral openings 124 in body 102 that may expose portions of channel 110 and/or channel loop 116 (as shown in FIGS. 1A and 2C). The proximal end 104 of body 102 is configured to receive an insertion tool wherein a distal portion of the tool releasably engages the anchor. In some embodiments, a shoulder 122 is configured at a distal end of the opening 112 and a proximal end of the channel 110 to interface with and engage with a distal portion of an insertion tool. The opening 112, proximal face 114, and/or shoulder 122 may be formed in any size and shape, and/or correlate to the size and shape of an insertion tool.

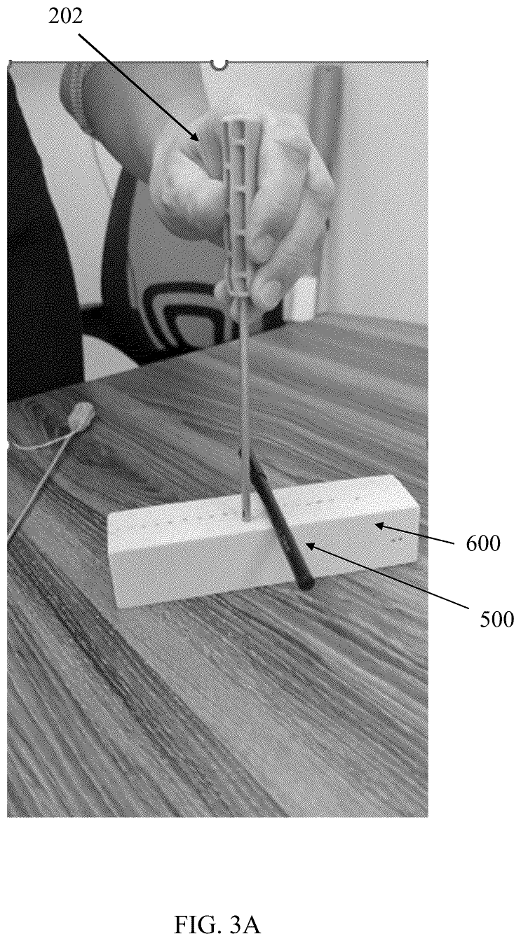

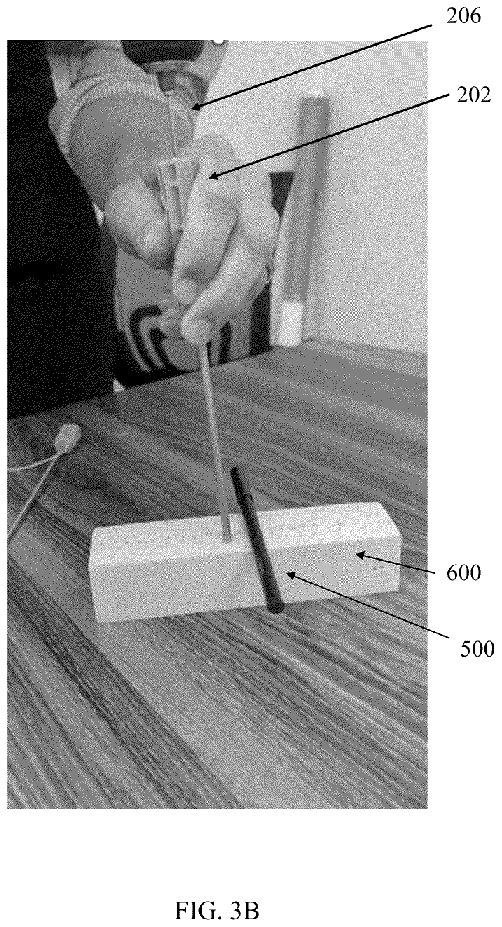

Aspects of a knotless anchor system 200 are shown in FIGS. 2A-2D. In some embodiments, system 200 comprises one or more suture anchor 100, a guide tool 202 and an insertion or inserter tool 204. In some embodiments, system 200 further comprises a drill tool 206. In some embodiments, guide tool 202 comprises a trocar 210, and/or one or more lateral opening 212 (e.g., viewing window). Guide tool 202, inserter tool 204 and drill tool 206 are used to secure a suture anchor 100 into bone near a target tissue such as a muscle or ligament. FIGS. 3A to 7B describe an exemplary method of using system 200 to place anchor 100 into bone for ligament repair.

Various methods for using anchor system 200 and/or anchor 100 are now described. A method for treating soft tissue includes passing tapered repair suture at least partially around the soft tissue, shuttling the tapered repair suture through the channel and the distal channel loop of an implanted anchor (e.g., suture anchor 100) by pulling the tapered repair suture with the shuttle suture, and cinching the tapered repair suture in a portion of the anchor. The soft tissue can be a labrum as shown in the specific examples herein, or any type of soft tissue.

Embodiments of the knotless anchor system use a preloaded shuttling suture and a tapered suture as a means of cinching to repair soft tissue, such as a torn labrum. Accordingly, in some embodiments, steps for treating a labrum include: positioning a guide tool with trocar at a target site, drilling an anchor hole with a drill tool, placing an inserter tool and an anchor (e.g., suture anchor 100) preloaded with at least one shuttle suture and/or tapered suture through the guide tool, impacting the inserter tool with anchor until the anchor is positioned and/or stops advancing, removing the guide tool and inserter tool, passing the tapered repair suture around the labrum, shuttling the tapered repair suture through the anchor with the shuttle suture, and cinching a portion of the tapered repair suture within the anchor. In some embodiments, the steps for treating a labrum further comprise tying or passing the tapered repair suture to one or more additional anchors.

In some embodiments, a method for repairing a labrum comprises the steps of providing at least one knotless suture anchor (e.g., suture anchor 100), preloading the anchor with a shuttle suture positioned through a distal channel loop in the anchor and extending out proximally from the anchor with first and second ends, wherein the first end comprises a loop, and the second end is a free end, implanting or securing the anchor in bone through a pre-drilled hole, passing the tapered repair suture around tissue (e.g., the labrum), passing a portion of the tapered repair suture through the loop of the shuttle suture, pulling the tapered suture through the anchor with the shuttle suture, releasing the shuttle suture from the tapered suture, capturing the tissue with the tapered suture by cinching the tapered suture within a channel of the anchor.

The shuttle suture is able to pull the small end of the tapered suture through the anchor, but as the tapered suture diameter increases, the fit is increasingly tighter in the anchor. This allows the user to cinch the labrum down without the suture backing up. In some embodiments, the shuttle suture may pull the tapered repair suture around the eyelet through a second hole extending longitudinally through the anchor or back through the same hole. In some embodiments, the anchor may also be loaded with a 3rd suture which can be used for a traditional tie-down repair. In some embodiments, the anchor is provided with a preloaded tapered repair suture which is secured to a distal eyelet in the anchor.

The described systems and methods are particularly suited for providing consistent and predictable suture tensioning characteristics and fixation strength for a knotless suture anchor. The friction fit of the tapered suture provides direct, predictable and intuitive tensioning characteristics with feedback that is easy to interpret. The disclosed suture anchors and systems may be used for various surgical procedures including orthopedic and soft tissue surgeries to anchor and secure soft tissue (such as tendons or ligaments) to bone. Each suture anchor provides a secure fixation point for sutures, which are used to reattach or repair damaged tissue. The disclosed anchors may be used in any procedures like rotator cuff repairs, labral repairs in the shoulder, Achilles tendon repairs, and ligament reconstructions.

The following publications are incorporated herein by reference in their entireties: U.S. Patent Publication No. US 2023/0248351 A1, U.S. Patent Publication No. US 2023/0157681 A1, and U.S. Patent Publication No. US 2024/0225631 A1.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

FIGS. 3A-7B depict exemplary steps for a method for using a knotless suture anchor system for labral repair utilizing a bone and soft tissue prototype. Steps for an exemplary method will now be described.

With reference now to FIGS. 3A & 3B, a guide with trocar is placed in the bone 600 (FIG. 3A) and a drill is inserted into the guide (FIG. 3B) for forming the anchor hole.

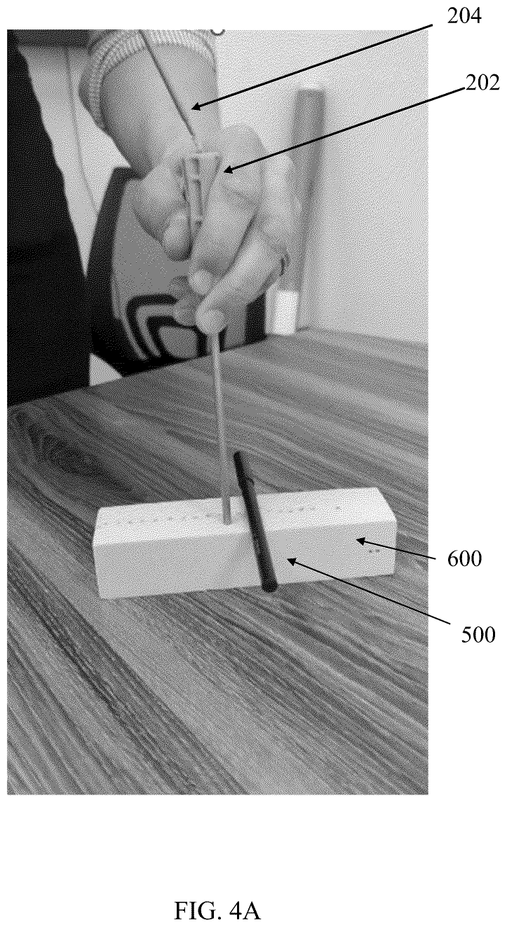

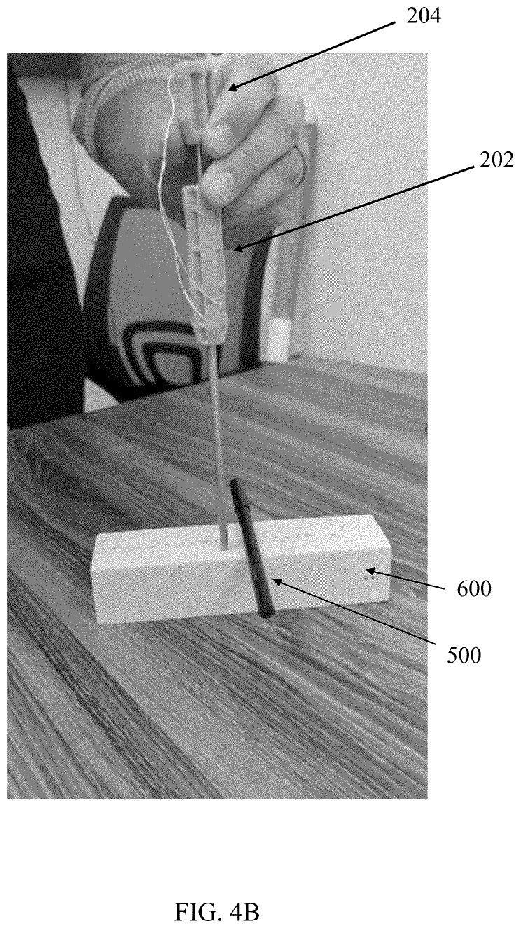

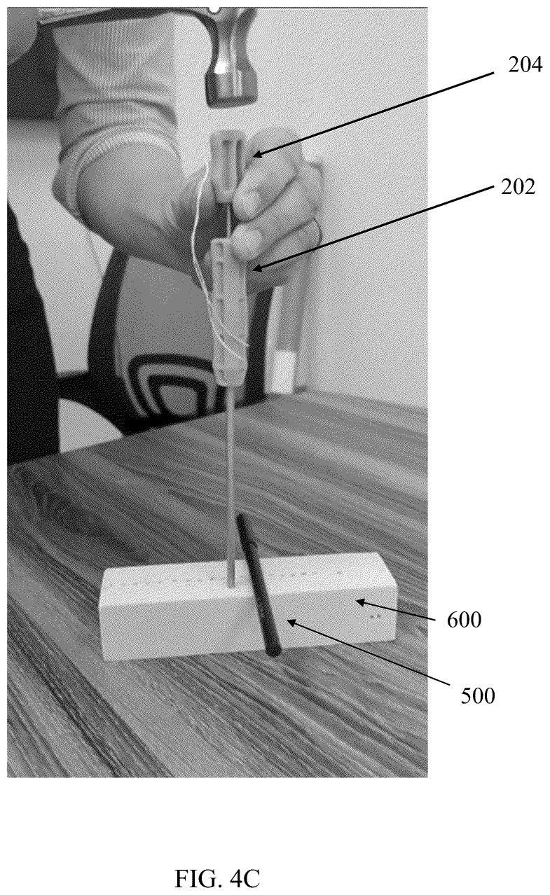

Next, as shown in FIGS. 4A-4C, the anchor inserter is inserted into the guide (FIGS. 4A & 4B), and the head of the anchor inserter is impacted until the anchor stops and is fully advanced into the bone hole (FIG. 4C).

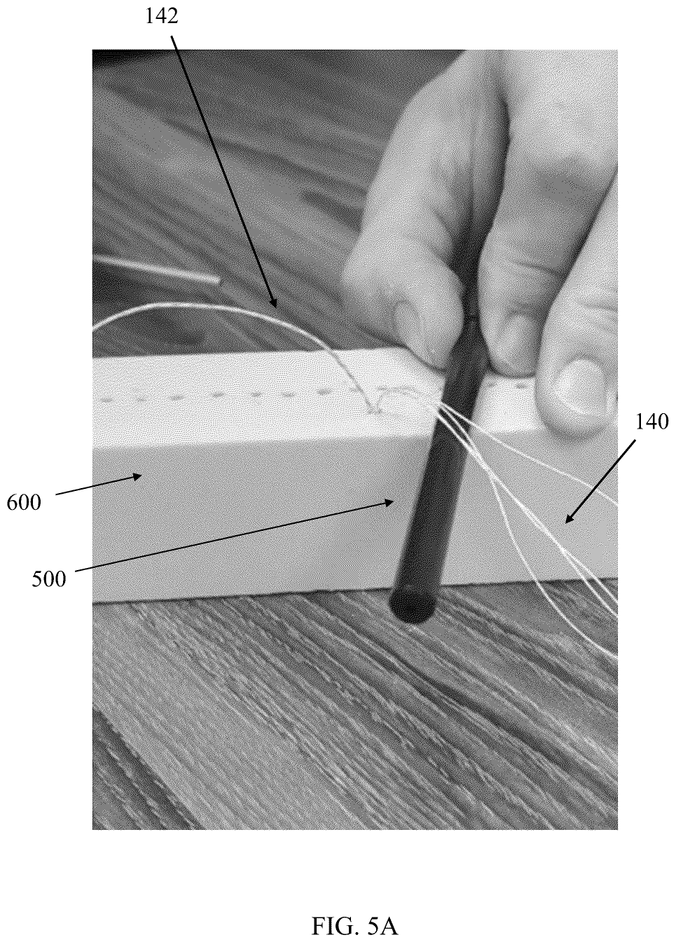

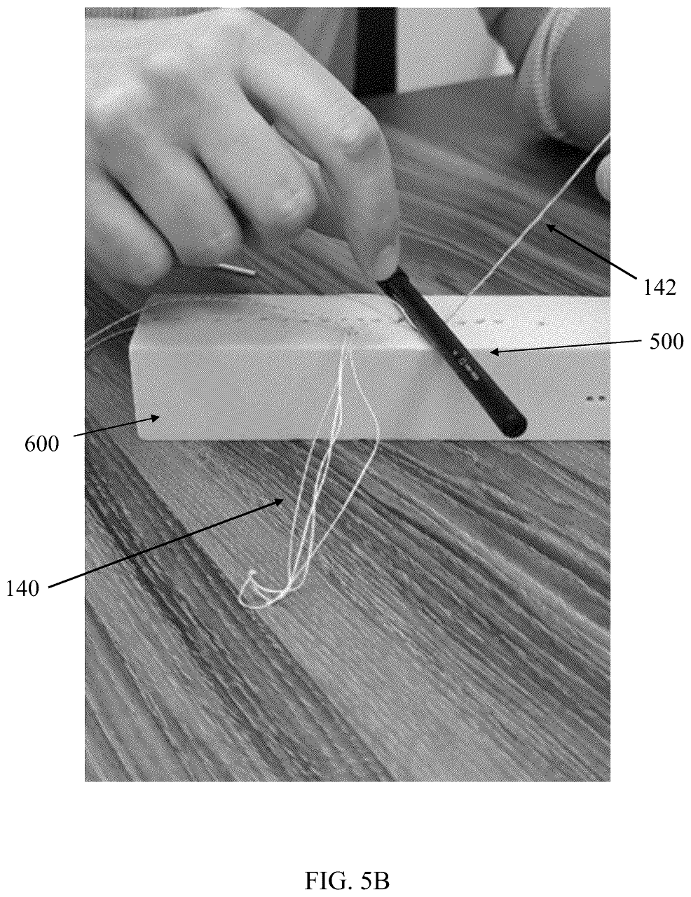

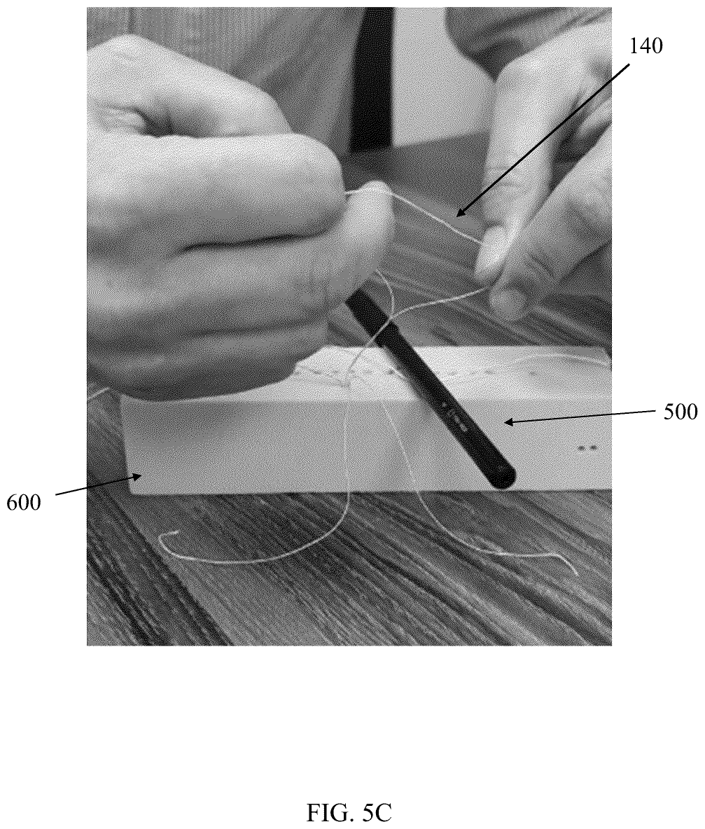

At this stage, as shown in FIGS. 5A-5C, both the anchor inserter and guide can be withdrawn, leaving the anchor in the hole and the tapered repair suture 142 and shuttle suture 140 exposed (FIG. 5A). The free end of the tapered repair suture 142 can pass below the labrum 500 (FIG. 5B). The free end of the tapered repair suture 142 is then fed into the loop of the shuttle suture (FIG. 5C).

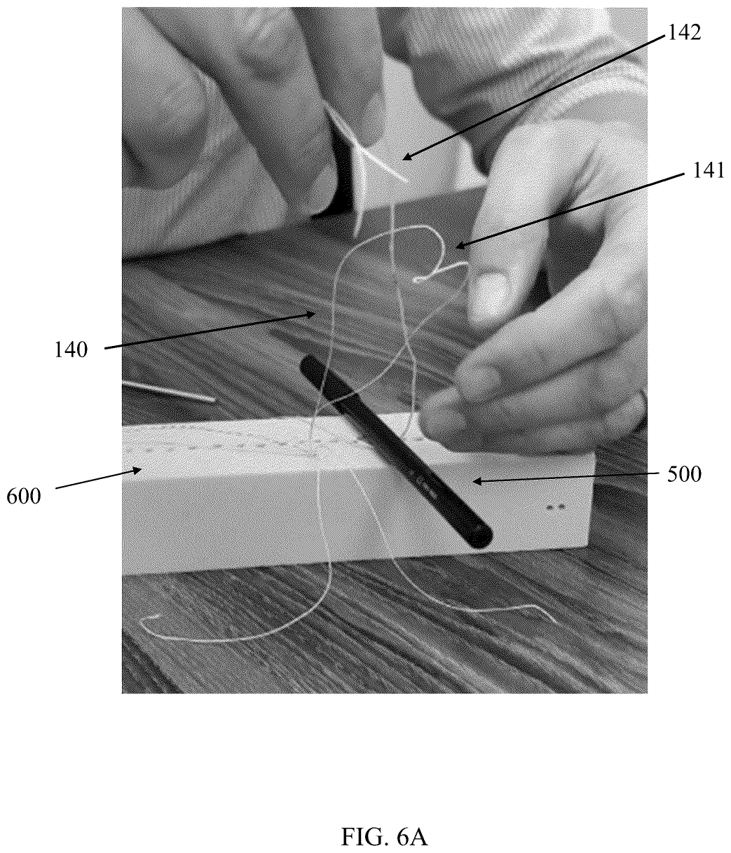

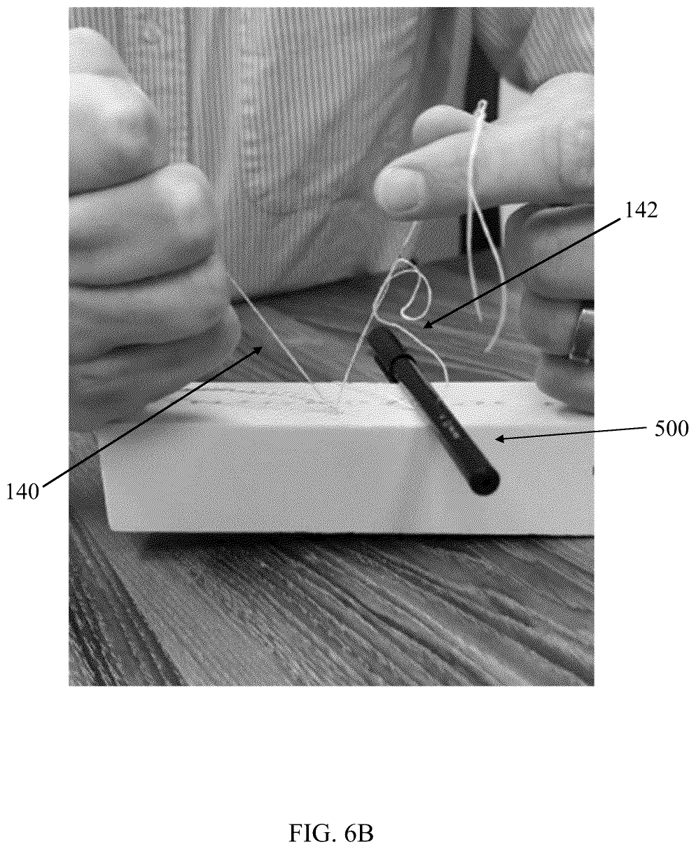

With reference now to FIGS. 6A & 6B, with the free end of the tapered repair suture is passed through the loop of the shuttle suture (FIG. 6A), pulling the free end of the shuttle suture will now shuttle the tapered repair suture through the anchor (FIG. 6B).

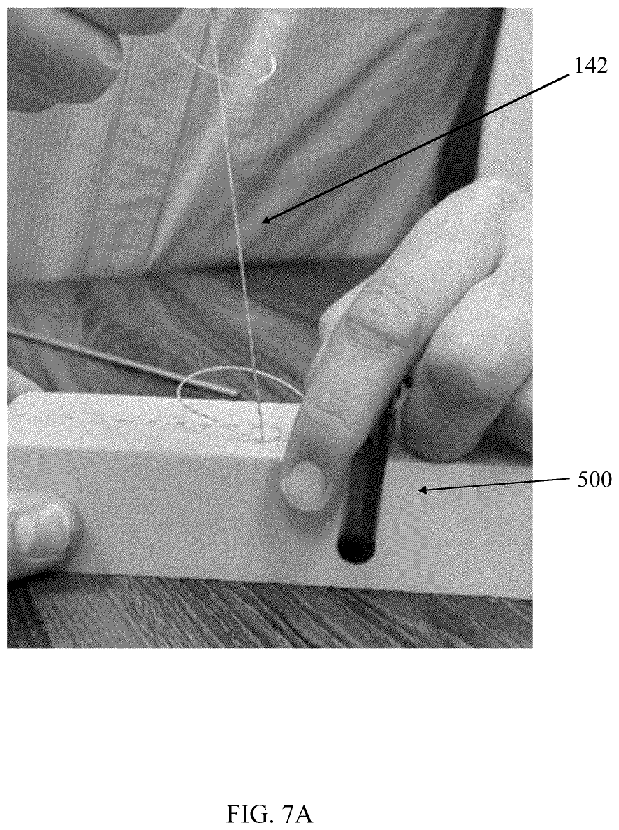

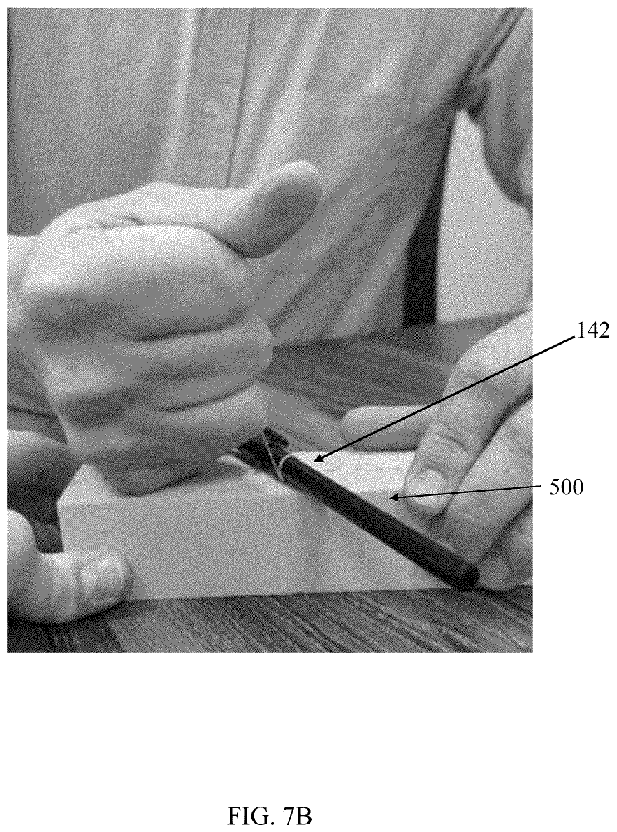

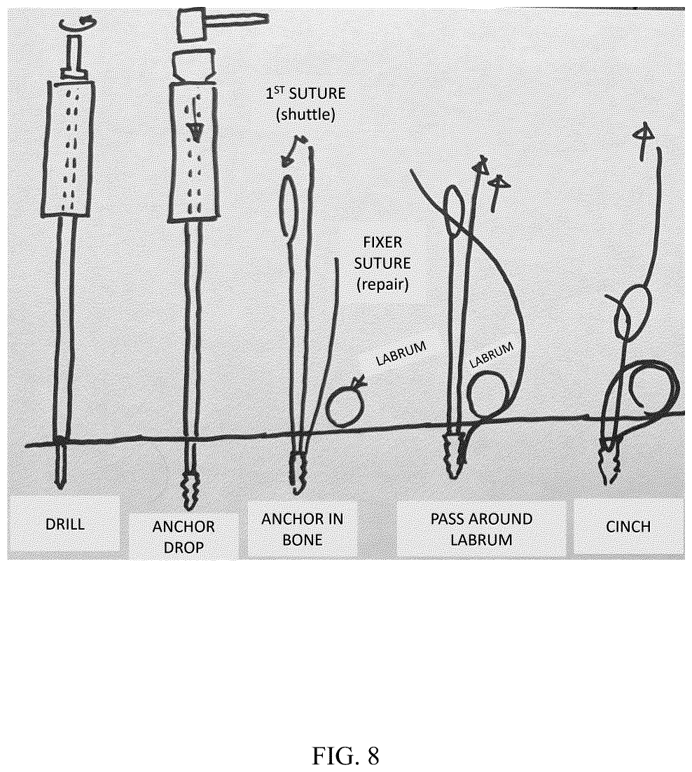

With reference now to FIGS. 7A & 7B, the tapered repair suture is looped around the labrum and through the anchor, thus pulling the tapered repair suture (FIG. 7A) cinches it into place. The taper is designed to arrest movement at this stage. A tight loop is formed (FIG. 7B) around the labrum, holding it securely against the anchor and bone.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A suture anchor system comprising:
at least one suture anchor comprising:
an elongate anchor body having at least one anchoring element, and
a channel having a distal channel loop and a proximal opening in a proximal face of the elongate anchor body; and
a tapered repair suture.

2. The suture anchor system of claim 1, wherein the tapered repair suture is configured to arrest advancement of the repair suture via an interface between a tapered portion of the tapered repair suture and the channel.

3. The suture anchor system of claim 1, wherein the at least one suture anchor comprises an eyelet extending through a portion of the anchor body.

4. The suture anchor system of claim 3, wherein the tapered repair suture is preloaded through the eyelet.

5. The suture anchor system of claim 3, wherein the tapered repair suture is affixed to the eyelet.

6. The suture anchor system of claim 1, further comprising at least one shuttle suture.

7. The suture anchor system of claim 6, wherein the at least one shuttle suture is preloaded through the distal channel loop of the suture anchor.

8. The suture anchor system of claim 7, wherein the at least one shuttle suture comprises a looped end and a free end, each extending out from the proximal opening of the anchor body.

9. The suture anchor system of claim 8, wherein the tapered repair suture releasably engages the looped end of the shuttle suture.

10. The suture anchor system of claim 1, wherein the at least one suture anchor comprises first and second suture anchors.

11. The suture anchor system of claim 1, wherein the tapered suture tapers in width along its length from about 0.3 mm to about 0.65 mm.

12. The suture anchor system of claim 11, wherein the tapered suture has a length ranging between 80 mm and 200 mm.

13. The suture anchor system of claim 1, further comprising:
a guide tool; and
an inserter tool, wherein the suture anchor engages the inserter tool, and the inserter tool extends through a portion of the guide tool to secure the suture anchor in a bone site.

14. The suture anchor system of claim 13, further comprising:
a drill tool, wherein the drill tool extends through a portion of the guide tool to drill a drill hole at the bone site.

15. A method for treating soft tissue, comprising:
providing the system of claim 1;
securing the suture anchor into a hole in bone;
passing the tapered repair suture at least partially around the soft tissue;
shuttling the tapered repair suture through the distal channel loop; and
cinching the tapered repair suture in the channel of the anchor, thereby capturing the soft tissue with the tapered repair suture.

16. The method of claim 15, wherein the tapered repair suture is affixed to the suture anchor prior to the securing step.

17. The method of claim 15, further comprising:
providing a second suture anchor;
securing the second anchor into a second hole in bone;
passing the tapered repair suture at least partially around the soft tissue;
shuttling the tapered repair suture through the distal channel loop of the second suture anchor, and
cinching the tapered repair suture in the channel of the second suture anchor, thereby capturing the soft tissue with the tapered repair suture.

18. A method for treating soft tissue, comprising:
providing the system of claim 5;
securing the suture anchor into a hole in bone;
passing the tapered repair suture at least partially around the soft tissue;
shuttling the tapered repair suture through the distal channel loop of the anchor with the shuttle suture; and
cinching the tapered repair suture in the channel of the suture anchor, thereby capturing the soft tissue with the tapered repair suture.

19. The method of claim 18, further comprising:
releasing the shuttle suture from the tapered repair suture.

20. The method of claim 18, further comprising:
providing a second suture anchor;
securing the second suture anchor into a second hole in bone;
passing the tapered repair suture at least partially around the soft tissue;
shuttling the tapered repair suture through the distal channel loop of the second suture anchor, and
cinching the tapered repair suture in the channel of the second suture anchor, thereby capturing the soft tissue with the tapered repair suture.
